(19) United States
(12) Patent Application Publication
(10) Pub. No.: US 2007/0134353 A1
(43) Pub. Date: Jun. 14, 2007

(54) PHYTOESTROGENIC COMPOSITION COMPRISING AN EXTRACT OF CHINESE LICORICE ROOT, ISOLIQUIRITIN OR LIQUIRITIN

(76) Inventors: Kyung Sun Kang, Seoul (KR); Jeong Chan Ra, Gyeonggi-do (KR); Eun Hye Jo, Seoul (KR)

Correspondence Address: INTELLECTUAL PROPERTY / TECHNOLOGY LAW, PO BOX 14329, RESEARCH TRIANGLE PARK, NC, 27709, US

(21) Appl. No.: 11/673,562

(22) Filed: Feb. 10, 2007

Related U.S. Application Data
(62) Division of application No. 11/018,363, filed on Dec. 20, 2004.

(30) Foreign Application Priority Data
Mar. 16, 2004 (KR) 10-2004-0017738

(51) Int. Cl. A61K 36/48; A61K 36/484; A61K 31/704

(52) U.S. Cl. 424/757; 514/33

(57) ABSTRACT

A phytoestrogen composition comprising an extract of Chinese licorice root (Glycyrrhiza uralensis Fisch), liquiritin or isoliquiritin isolated therefrom, which show estrogen activity and inhibitory effect on proliferation of cancer cells, as an effective ingredient. The Chinese licorice root extract, as well as liquiritin or isoliquiritin isolated therefrom, show estrogenicity but not androgenicity, suppress activity of the endocrine disruptor bisphenol-A and inhibit proliferation of cancer cells, e.g., breast cancer cells. The composition is usefully employed as a phytoestrogen for hormone replacement therapy and/or prevention and treatment of breast cancer.

### CROSS-REFERENCE TO RELATED APPLICATION

This is a division of U.S. patent application Ser. No. 11/018,363 filed Dec. 20, 2004, which in turn claims priority under 35 USC 119 of Korean Patent Application No. 10-2004-0017738 filed Mar. 16, 2004. Priority of U.S. patent application Ser. No. 11/018,363 is hereby claimed under the provisions of 35 USC 120. The disclosure of such applications is hereby incorporated by reference, in their respective entireties.

### BACKGROUND OF THE INVENTION

1. Field of the Invention

The present invention relates to a phytoestrogen composition comprising an extract of Chinese licorice root (Glycyrrhiza uralensis Fisch), liquiritin or isoliquiritin isolated therefrom, which show estrogen activity and anti-tumor activity, as an effective ingredient.

2. Background of the Related Art

Recently, phytoestrogen, a vegetable-originated estrogen, has attracted public attention as a substitute for a synthetic estrogen, since the synthetic estrogen may cause cancer or cardiovascular diseases in hormone replacement therapy (HRT), which is widely used as a method for treatment of menopausal symptoms. Typical types of phytoestrogen include lignans and isoflavons. Vegetable oil, field grain, vegetables, pulse, and fruit are main sources of supply for lignans, and beans and pulse products are a source of supply for isoflavons.

The important functions of such phytoestrogen is to obstruct the action of enzymes to multiply cancer cells, and to promote division of normal cells so as to prevent breast cancer and prostate cancer (Horn-Ross, P. L. et al., J. Nat. Cancer Inst., 95:1158, 2003; Watanabe, S. et al., Gan To Kagaku Rhoho, 30:902, 2003; Peeters, P. H. et al., Breast Cancer Res. Treat., 77:171, 2003; and U.S. Pat. No. 6,265,448). Also, it has been known that phytoestrogen is helpful for prevention and treatment of osteoporosis, cardiovascular diseases and menopausal symptoms.

Chinese licorice root, a leguminous perennial plant, is a medicinal herb grown in Siberia, Mongolia and Northern China. Chinese licorice root extract is used as a purified component of glycyrrhizic acid, glycyrrhetic acid, liquiritin, isoliquiritin and the like. Particularly, it serves as a natural antibiotic showing excellent medicinal effect in heat rashes and acne and has antiphlogistic action as an antiinflammation agent.

### SUMMARY OF THE INVENTION

The present invention relates to a novel phytoestrogen having inhibitory effect on proliferation of cancer cells and at the same time showing estrogen activity, and to compositions comprising same.

In one aspect, the present invention relates to a phytoestrogen composition comprising an extract of Chinese licorice root (Glycyrrhiza uralensis Fisch), liquiritin or isoliquiritin isolated therefrom as an effective ingredient.

In a particular aspect, the invention relates to a phytoestrogen composition comprising an organic solvent extract of Chinese licorice root (Glycyrrhiza uralensis Fisch).

In another aspect, the invention relates to a method of prevention or treatment of a physiological condition selected from the group consisting of osteoporosis, cardiovascular diseases, menopausal symptoms and cancers, comprising administering to a subject in need thereof, an effective amount of a phytoestrogen composition comprising an organic solvent extract of Chinese licorice root (Glycyrrhiza uralensis Fisch).

In yet another aspect, the present invention relates to a health-promoting food for prevention of menopausal symptoms and cancers, comprising an extract of Chinese licorice root, liquiritin or isoliquiritin isolated therefrom as an effective ingredient.

In a particular aspect, the invention relates to a health-promoting food for prevention of menopausal symptoms and cancers, comprising an organic solvent extract of Chinese licorice root (Glycyrrhiza uralensis Fisch).

In a further aspect, the invention relates to a phytoestrogen composition comprising liquiritin or isoliquiritin.

Another aspect of the invention relates to a method for prevention or treatment of a physiological condition selected from the group consisting of osteoporosis, cardiovascular diseases, menopausal symptoms and cancers, comprising administering to a subject in need thereof, an effective amount of a phytoestrogen composition comprising liquiritin or isoliquiritin.

A still further aspect of the invention relates to a health-promoting food for prevention of menopausal symptoms and cancers, comprising liquiritin or isoliquiritin.

Other aspects, features and advantages of the invention will be more fully apparent from the ensuing disclosure and appended claims.

### BRIEF DESCRIPTION OF THE DRAWINGS

FIG. 1 shows illustrative procedures to isolate liquiritin and isoliquiritin from an extract of Chinese licorice root.

FIG. 2 shows estrogen responses (ER) of Chinese licorice root extracts (a) and isoliquiritin (b), as measured by yeast assay.

FIG. 3 shows androgen responses (AR) of Chinese licorice root extracts, as measured by yeast assay.

FIG. 4 shows inhibitory effects of extracts of Chinese licorice root, extracted at various concentrations of EtOH, on MCF-7 breast cancer cells and bisphenol A.

FIG. 5 shows inhibitory effects of isoliquiritin, at various concentrations, on MCF-7 breast cancer cells.

FIG. 6 shows inhibitory effects of Chinese licorice root extracts, prepared with various solvents, on MCF-7 breast cancer cells and bisphenol A.

FIG. 7 shows cytotoxicity of Chinese licorice root extracts, prepared with various solvents, on MCF-7 breast cancer cells.

FIG. 8 shows incision sites of the uterus and the vagina for histopathological study.

FIG. 9 shows changes in body weight during administration of Chinese licorice root extracts and bisphenol A.

FIG. 10 shows absolute weights of the uterus after treatment with Chinese licorice root extracts.

FIG. 11 shows relative weights of the uterus after treatment with Chinese licorice root extracts.

FIG. 12 shows absolute weights of the vagina after treatment with Chinese licorice root extracts.

FIG. 13 shows relative weights of the vagina after treatment with Chinese licorice root extracts.

FIG. 14 shows tissue photographs of the uterine epithelial cell.

FIG. 15 shows tissue photographs of the vaginal epithelial cell.

### DETAILED DESCRIPTION OF THE INVENTION, AND PREFERRED EMBODIMENTS THEREOF

The present invention reflects the finding by the present inventors of a novel phytoestrogen and the discovery that an extract of Chinese licorice root, as well as liquiritin or isoliquiritin isolated therefrom, has inhibitory effect on proliferation of cancer cells, e.g., breast cancer cells, and at the same time, shows estrogen activity.

The present invention thus is directed to a phytoestrogen composition comprising an extract of Chinese licorice root (Glycyrrhiza uralensis Fisch), liquiritin or isoliquiritin isolated therefrom as an effective ingredient.

The Chinese licorice root extract according to the present invention can be prepared as follows. Chinese licorice root or a dehydrated product thereof is powdered and extracted with a suitable solvent, such as for example C₁-C₄ alcohol, an aqueous solution of a C₁-C₄ alcohol, ethyl acetate (EtOAc), chloroform (CHCl₃), or hexane, of about 1 to 100 fold, preferably about 10 to 50 fold volume of Chinese licorice root sample weight, by any known extraction method, such as hot water extraction at 60˜90° C., cold percolation extraction, reflux cooling extraction or ultrasonic extraction, with reflux cooling extraction being a preferred mode of extraction. The extraction process typically is repeated for a suitable number of times, e.g., 1˜5 time(s), and preferably 1˜3 time(s). The collected supernatant is condensed at reduced pressure and dried. A preferred C₁-C₄ alcohol for extraction is methanol or ethanol.

Liquiritin and isoliquiritin according to the present invention can be isolated from the Chinese licorice root extract, prepared as described above or otherwise obtained through a chemical synthesis thereof. In one preferred embodiment of the invention, the ethanol, methanol, ethyl acetate, chloroform or hexane extract, preferably ethanol extract, prepared as described above, is subjected to silica gel column chromatography and reverse phase column chromatography to obtain liquiritin and isoliquiritin.

The inventive Chinese licorice root extract, as well as liquiritin or isoliquiritin isolated therefrom, show estrogenicity but do not show androgenicity. Additionally, they suppress the activity of bisphenol-A, which is an endocrine disrupter, and inhibit proliferation of cancer cells including breast cancer cells. Therefore, they can be usefully used as a phytoestrogen for hormone replacement therapy and prevention and treatment of breast cancer.

In one evaluation of the anticancer agents of the present invention, the Chinese licorice root extract at a concentration of 100 μg/ml, and liquiritin and isoliquiritin at a concentration of 100 μg/ml, inhibited proliferation of MCF-7 breast cancer cell line. More specifically, the hexane, CHCI₃ and EtOAc extracts of Chinese licorice root showed proliferation inhibiting effect even at a low concentration of 10 μg/ml.

The Chinese licorice root extract, or liquiritin or isoliquiritin isolated therefrom, may be mixed with a pharmaceutically acceptable carrier or excipient or diluted in a diluent to prepare a pharmaceutical composition having the foregoing functions, according to conventional methods. Examples of suitable carrier, excipient and diluent species include lactose, dextrose, sucrose, sorbitol, mannitol, xylitol, erythritol, maltitol, starch, acacia rubber, alginates, gelatin, calcium phosphate, calcium silicate, cellulose, methyl cellulose, amorphous cellulose, polyvinyl pyrrolidone, water, methyl hydroxybenzoate, propyl hydroxybenzoate, talc, magnesium stearate and mineral oil.

The pharmaceutical composition may further comprise a filler, an anti-coagulant agent, a lubricant, a wetting agent, a flavor, an emulsifying agent, a preservative and the like. The pharmaceutical composition can be formulated using well-known methods, to provide rapid, sustained or delayed release of an active ingredient after administration to an animal subject, e.g., a mammal, such as human, horse, sheep, cow, pig, etc. in need thereof for treatment or prophylaxis of a disease state or physiological condition for which the pharmaceutical composition of the invention is effective. The formulation may be in any suitable form, e.g., in a form such as tablet, pill, powder, sachet, elixir, suspension, emulsion, solution, syrup, aerosol, soft or hard gelatin capsule, sterile injection, sterile powder or the like.

The pharmaceutical composition according to the present invention can be administered via any suitable mode of administration, including various routes such as oral, transdermal, subcutaneous, intravenous or intramuscular administration. The normal daily dose of the inventive Chinese licorice root extract is in a range of 1 to 500 mg/kg body weight, preferably 10 to 100 mg/kg body weight, and may be administered once daily or several times daily in divided doses. The normal daily dose of the liquiritin and isoliquiritin is in a range of 0.1 to 50 mg/kg body weight, preferably 1 to 10 mg/kg body weight, and may be administered once daily or several times daily in divided doses. It will nonetheless be understood that the actual dose of the active ingredient may be widely varied to good effect in the broad practice of the invention, and is determined in view of various relevant factors such as administration route, age, sex and body weight of the patient, and severity of the disease, and thus the foregoing dose range and dose regimen is not intended to limit the scope of the invention in any way. The Chinese licorice root extract, liquiritin or isoliquiritin may be administered as individual therapeutic agents, or they may be administered in any suitable combinations of two or more of such active ingredients, e.g., as a combination of liquiritin and isoliquiritin, or as a liquiritin-enriched Chinese licorice root extract mixture, or as an isoliquiritin-enriched Chinese licorice root extract mixture, or as a liquiritin-enriched and isoliquiritin-enriched Chinese licorice root extract mixture.

The inventive Chinese licorice root extract, liquiritin or isoliquiritin isolated therefrom may be added to food or beverage for the purpose of health promotion by phytoestrogen activity and prevention of cancers. Thus, the present invention contemplates a food or beverage for health promotion for prevention of climacteric complaints and cancers, in which the food comprises the Chinese licorice root extract, liquiritin or isoliquiritin isolated therefrom according to the present invention, as an effective ingredient.

According to one aspect of the present invention, with respect to the content of the extract in a food or beverage, the extract may be typically added to a health-promoting food in an amount of 0.1 to 50% by weight, preferably 0.1 to 1% by weight, based on the total weight of the food, and may be added to a healthy beverage in an amount of 0.1 to 50 g, and preferably 0.1 to 1 g, based on 100 ml of the beverage. Also, liquiritin and isoliquiritin may be typically added to a health-promoting food in an amount of 0.01 to 5% by weight, preferably 0.01 to 0.1% by weight, based on the total weight of the food, and may be added to a healthy beverage in an amount of 0.01 to 5 g, and preferably 0.01 to 0.1 g, based on 100 ml of the beverage.

Providing that the healthy beverage composition of the present invention comprises the Chinese licorice root extract, liquiritin or isoliquiritin as an essential ingredient, there is no particular limitation in the other liquid components of such beverage, and the composition may further comprise one or more additives, such as various flavors or natural carbohydrates which are commonly used in beverages.

Examples of natural carbohydrates for such purpose include, without limitation, common sugars such as monosaccharides, for example, glucose, fructose and the like; disaccharides, for example, maltose, sucrose and the like; and polysaccharides, for example, dextrine, cyclodextrine and the like, and sugar alcohols such as xylitol, sorbitol, erythritol and the like. In addition to the foregoing, as the flavors, natural flavors (thaumatin, Stevia extract (for example, Rebaudioside A, glycyrrhizin and the like), and synthetic flavors (saccharine, aspartame and the like) may be advantageously used.

Further, beverages prepared according to the present invention may further contain various nutrients, vitamins, minerals (electrolytes), seasonings (artificial seasonings and natural seasonings), coloring agents and improving agents (cheese, chocolate and the like), pectic acid and salts thereof, alginic acid and salts thereof, organic acids, protective colloid thickeners, pH controllers, stabilizers, preservatives, glycerin, alcohols, carbonating agents used in carbonated beverages, and the like. These additives may be used independently or in combination.

### EXAMPLES

The present invention will hereinafter be described in further detail with reference to examples. It will however be obvious to a person skilled in the art that these examples can be modified into various different forms and the present invention is not limited to or by the examples. These examples are presented to further illustrate the present invention.

### Example 1

### Preparation of Chinese Licorice Root Extract

Chinese licorice root was mixed with 80% EtOH by 2 L of 80% EtOH per 100 g of Chinese licorice root, subjected to a primary extraction using a reflux condenser at 70˜80° C. for 3 hours, cooled and filtered through a filter paper (Whatman No. 1 filter). As needed, the residue on the filter paper was subjected to a secondary extraction under the same conditions as used for the primary extraction. The filtrate was concentrated to remove EtOH and lyophilized.

Meanwhile, Chinese licorice root was extracted with CHCl₃, hexane, 70% MeOH, EtOAc, water, 20, 40 and 60% aqueous EtOH solutions and 100% EtOH and the extracts were lyophilized, following the same procedures as described above.

100 mg of each of the lyophilized extracts was dissolved in 1 ml of 80% EtOH to prepare a 100 mg/ml stock solution. The stock solution was then diluted with 80% ethanol to a desired concentration, as needed.

### Example 2

### Isolation of Liquiritin and Isoliquiritin from Chinese Licorice Root Extract

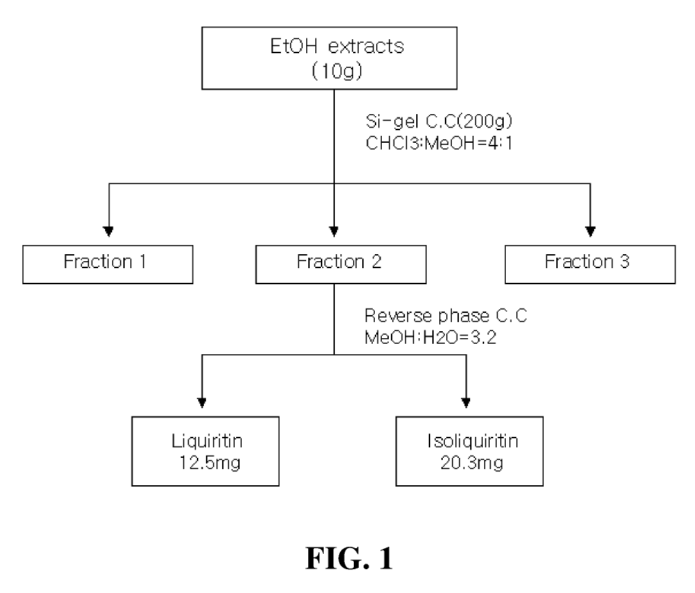

The EtOH phase extract prepared from Example 1 was subjected to chromatographic separation including silica gel column chromatography (eluent, CHCl₃: MeOH=4:1) and reverse phase column chromatography (eluent, MeOH : H₂O=3:2) to obtain compounds of white powder and yellow powder (FIG. 1). ¹H-NMR and ¹³C-NMR were performed to determine structures of the compounds. The ¹H-NMR and ¹³C-NMR data are shown in Table 1 and 2. From the NMR data of Table 1 and 2, it was confirmed that the compounds isolated in these Examples were liquiritin and isoliquiritin.

TABLE 1

| No of C | ¹H-NMR | ¹³C-NMR |
|---|---|---|
| C₂ | 5.18(1H, dd, J=3.0, 12.6 Hz) | 79.6 |
| C₃ | 2.74(1H, dd, J=3.0, 16.8 Hz) | 43.9 |
| | 3.05(1H, m) | |
| C₄ | | 192.1 |
| C₅ | 7.74(1H, d, J=8.7 Hz) | 128.8 |
| C₆ | 6.50(1H, dd, J=2.4, 8.7 Hz) | 110.8 |
| C₇ | | 165.8 |
| C₈ | 6.39(1H, d, J=2.4 Hz) | 102.8 |
| C₉ | | 164.3 |
| C₁₀ | | 114.0 |
| C_{1′} | | 133.4 |
| C_{2′} | 7.45(1H, dd, J=1.8, 12.0 Hz) | 127.5 |
| C_{3′} | 7.15(1H, dd, J=1.8, 12.0 Hz) | 116.8 |
| C_{4′} | | 158.2 |
| C_{5′} | 7.15(1H, dd, J=1.8, 12.0 Hz) | 116.8 |
| C_{6′} | 7.45(1H, dd, J=1.8, 12.0 Hz) | 127.5 |
| Glu C_{1″} | 4.95(1H, d, J=7.8 Hz) | 101.2 |
| C_{2″} | 3.42-5.50(1H, m) | 73.9 |
| C_{3″} | 3.42-5.50(1H, m) | 77.0 |
| C_{4″} | 3.42-5.50(1H, m) | 70.4 |
| C_{5″} | 3.42-5.50(1H, m) | 77.1 |
| C_{6″} | 3.90(1H, dd, J=1.8, 12 Hz) | 61.5 |
| | 3.70(1H, dd, J=5.1, 12.3 Hz) | |

Liquiritin (white powder)

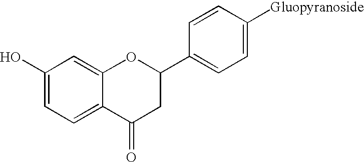

TABLE 2

| No of C | ¹H-NMR | ¹³C-NMR |
|---|---|---|
| C₁ | | 129.5 |
| C₂ | 7.73(1H, d, J=8.7 Hz) | 130.3 |
| C₃ | 7.18(1H, d, J=8.7 Hz) | 117.0 |
| C₄ | | 160.0 |
| C₅ | 7.18(1H, d, J=8.7 Hz) | 117.0 |
| C₆ | 7.73(1H, d, J=8.7 Hz) | 130.3 |
| C_{1′} | | 113.7 |
| C_{2′} | | 166.5 |
| C_{3′} | 6.32(1H, d, J=2.4 Hz) | 102.8 |
| C_{4′} | | 166.5 |
| C_{5′} | 6.44(1H, dd, J=2.4, 9.0 Hz) | 108.2 |
| C_{6′} | 8.01(1H, d, J=9.0 Hz) | 132.4 |
| α | 7.82(1H, d, J=15.3 Hz) | 119.1 |
| β | 7.70(1H, d, J=15.3 Hz) | 143.8 |
| β′ | | 192.4 |
| Glu C_{1″} | 5.00(1H, d, J=7.5 Hz) | 100.8 |
| C_{2″} | 3.39-3.52(1H, m) | 73.7 |
| C_{3″} | 3.39-3.52(1H, m) | 76.9 |
| C_{4″} | 3.39-3.52(1H, m) | 70.2 |
| C_{5″} | 3.39-3.52(1H, m) | 77.3 |
| C_{6″} | 33.94(1H, dd, J=2.1, 12.0 Hz) | 61.4 |
| | 3.74(1H, m) | |

Isoliquiritin (yellow powder)

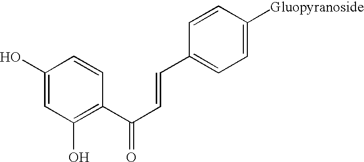

### Experimental Example 1

### Yeast Assay

In order to confirm that the Chinese licorice root extract, isoliquiritin and liquiritin prepared in Examples 1 and 2 show hormone activity, the extract was applied to recombinant yeast expressing human estrogen or androgen receptor to examine hormone production.

Saccharomyces cerevisiae ER+LYS 8127, supplied by Dr. Donald P. McDonnell of Duke University Medical Center, Durham, N.C., USA, which are recombinant yeast cells comprising human estrogen receptor (hER) or androgen receptor (hAR) gene and a vector expressing β-gal gene as a reporter were placed in a selective growth medium (Sigma, USA), containing yeast nitrogen substrate (without amino acid, 67 mg/ml), 1% dextrose, L-lysine (36 μg/ml) and L-histidine (24 μg/ml) and grown in a shaking incubator (200 rpm). The cultured yeasts were diluted in a medium to a proper concentration and 500 μM of CuSO₄ (Sigma, USA) was added thereto. The diluted yeasts were then aliquoted into a 50 Ml tube at a proper level. The experiment groups for yeast estrogen response (YER) and the experiment groups for yeast androgen response (YAR) were treated with 0.1% by weight of 100% EtOH Chinese licorice root extract with concentrations of 100 μg/ml and 1 mg/ml, prepared in Example 1, and isoliquiritin with concentrations of 10 μg/ml and 100 μg/ml, isolated in Example 2. Control group was treated with dimethyl sulfoxide (Sigma, USA). Also, as positive control groups in estrogenicity assay and androgenicity assay, the yeasts were treated with 10⁻⁹ M estradiol and nor-testosterone dissolved in DMSO.

The experimental groups and control groups treated with the respective materials were then cultured for 18 hours in a shaking incubator (200 rpm). The culture broth of each tube was diluted to the same concentration of 100 μg/ml and 1 mg/ml and each 100 μl of diluted product was placed in each well of 96-well microtiter plate (Nunc, Netherland). To each well, 100 μl of a Z buffer containing 2 mg/Ml o-nitrophenyl -β-D-galactopyranoside (Sigma, USA), 0.1% lauryl sulfate (Sigma, USA), 50 mM β-mercaptoethanol (BDH chemical LTD, England) and 200 U/Ml oxalyticase (Enzogenetics, USA) was added. After 20 minutes, the coloration was examined using a microplate reader at a wavelength of 420 nm.

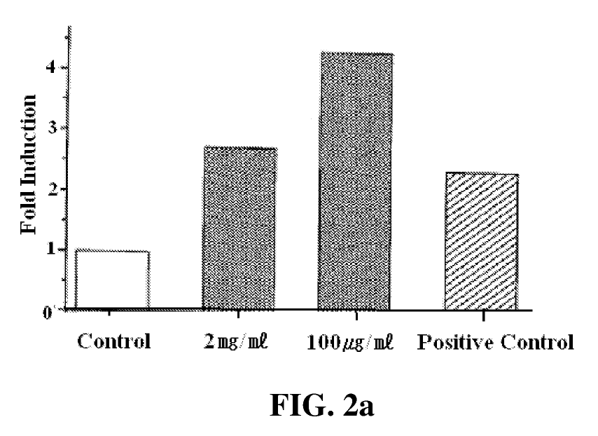

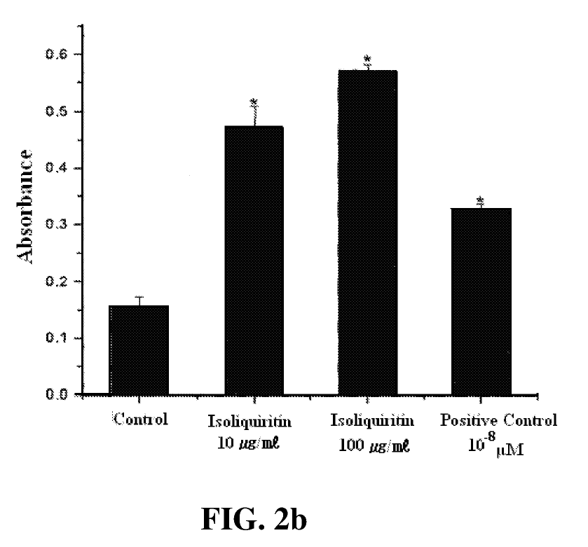

All the data were analyzed by ANOVA using the statistical program SAS and Dunnett's t-test was performed at significance level P<0.05. The estrogen responses (ER) and androgen responses (AR) of the Chinese licorice root extract and isoliquiritin, measured by the above-described method, are shown in FIG. 2a, FIG. 2b and FIG. 3.

As shown in FIG. 2a, the Chinese licorice root extract showed the highest estrogenicity at 100 μg/ml of concentration and showed concentration-dependent estrogenicity at 100 μg/ml and 1 mg/ml of concentrations. The estrogenicities at these concentrations were higher than that of the positive control estradiol. As shown in FIG. 2b, isoliquiritin showed the highest estrogenicity at 10 μg/ml of concentration and showed concentration-dependent estrogenicity at 10 μg/ml and 100 μg/ml of concentrations. The estrogenicities at these concentrations were higher than that of the positive control estradiol. When liquiritin was used instead of isoliquiritin, similar results were obtained.

Additionally, from the results of the androgenicity response in FIG. 3, it was confirmed that the Chinese licorice root extract at 1 mg/ml of concentration showed androgenicity lower than nor-testosterone which had been used as the positive control group but higher than the control group, while the extract at 100 μg/ml of concentration did not show androgenicity.

### Experimental Example 2

### Assay of Inhibitory Effect on MCF-7 Cell Line Proliferation and Bisphenol-A (BPA) Activity

In order to assay the inhibitory effect of the Chinese licorice root extracts, liquiritin and isoliquiritin prepared in Examples 1 and 2 on proliferation of MCF-7 cell line and bisphenol-A activity, the following experiment was performed.

5×10⁴ cells/ml of MCF-7 cells (ATCC HTB-22) were placed in a 6-well culture plate and allowed to be attached for 24 hours. Phenol red-free D-medium supplemented with 5% fetal bovine serum (FBS) and 3 Ml/l PSN antibiotic mixture (Gibco, USA) was added thereto, followed by incubation in an incubator kept at 5% CO₂, 95% air, 100% humidity, 37° C. for 24 hours.

After incubation for 24 hours, the medium was removed and the cultured cells were incubated in phenol red-free D-medium supplemented with 5% DCC-FBS (dextran-coated charcoal-stripped FBS; Hyclone, USA) and 3 ml/l PSN antibiotic mixture (test medium), in a medium to which 1% by weight of various Chinese licorice root extracts (10 μg/ml, 10 μg/ml or 1 mg/ml of concentrations) prepared in Example 1 were added, or in a medium to which liquiritin or isoliquiritin isolated in Example 2 were added at the concentrations of 100 μg/ml, 200 μg/ml, 300 μg/ml, 400 μg/ml or 500 μg/ml, at 37° C. for 3 days. During the incubation, the test media were changed once. After 72 hours, the cells were treated with 1 Ml of 0.1 N NaOH (Sigma Co.) and collected to examine DNA content with a spectrophotometer at OD 260 nm.

Additionally, in order to examine if the Chinese licorice root extract can suppress activity of bisphenol-A (BPA), which is an endocrine disruptor having tumor-inducing activity, in the foregoing experiment, BPA was used instead of the Chinese licorice root extract or the Chinese licorice root extract and BPA were used simultaneously. Here, BPA treated at the concentration (16 μg/ml) showed the highest effect on proliferation of MCF-7 cells in the pretest. In the experimental group treated with both the Chinese licorice root extract and BPA, the Chinese licorice root extract was concentrated to half the volume of Chinese licorice root single treatment group so that the treating agent had the same volume as that of the Chinese licorice root single treatment group.

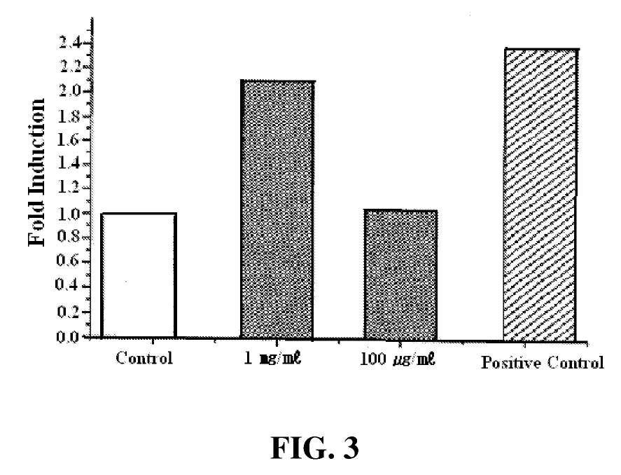

FIG. 4 shows inhibitory effect of extracts of Chinese licorice root extracted at various concentrations of EtOH on MCF-7 breast cancer cells and bisphenol A. In the MCF-7 cells incubated with the extracts of Chinese licorice root extracted with EtOH at a concentration of 0 to 100%, the proliferation inhibiting effect increased as the concentration of ethanol increased. The Chinese licorice root EtOH 0% extract (water extract) did not have inhibitory effect on MCF-7 breast cancer cells and bisphenol A.

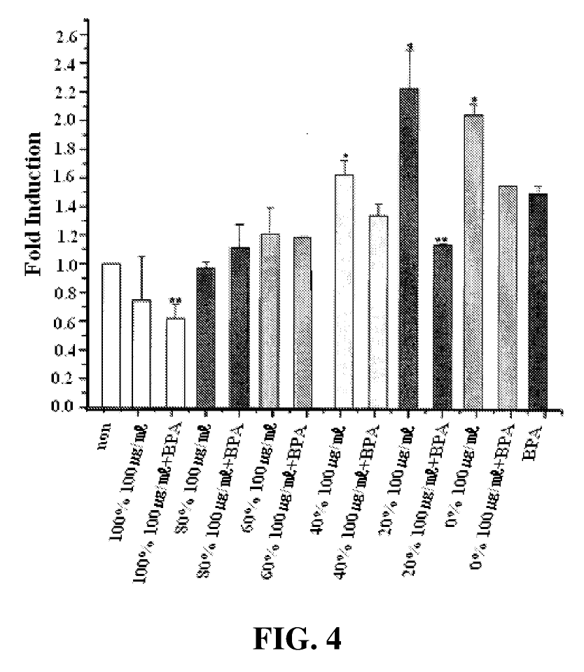

FIG. 5 shows inhibitory effect of isoliquiritin at various concentrations on MCF-7 breast cancer cells. When isoliquiritin was used at a concentration of 100 μg/ml, no significant proliferation of breast cancer cells was observed. Particularly, isoliquiritin at a concentration of 300 μg/ml showed excellent inhibitory effect on proliferation of breast cancer. When liquiritin was used instead of isoliquiritin, similar effects were observed.

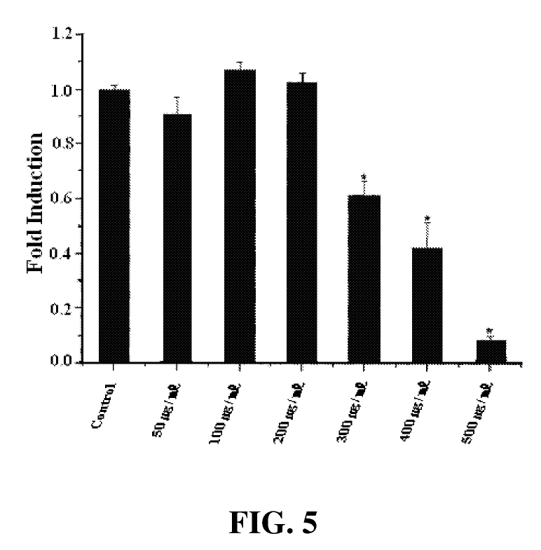

FIG. 6 showed inhibitory effect of Chinese licorice root extracts in various solvents on MCF-7 breast cancer cells and bisphenol A, in which the Chinese licorice root extracts which had been extracted with hexane, EtOAc, CHCI₃, 70% MeOH, respectively, were diluted 10³ times and 10⁴ times in 80% EtOH to be used at a concentration of 10 μg/ml or 100 μg/ml. Here, the test substances were diluted 10⁴ times, since they showed cytotoxicity such as cell transformation at a dilution of 100 times and 1000 times. As shown in FIG. 6, the Chinese licorice root extracts at a concentration of 10˜100 μg/ml prepared using various solvents showed inhibitory effects on MCF-7 breast cancer cells and bisphenol A.

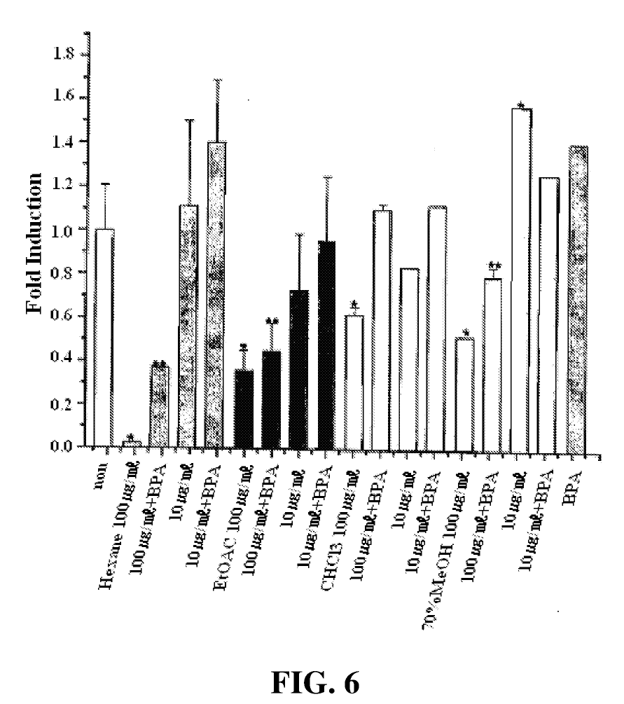

Meanwhile, when the above described experiment was repeated using the Chinese licorice root extract at a concentration of 1 mg/ml which had been diluted 100 times, it was impossible to measure absorbance since the concentration of the extract was too high. Therefore, anti-proliferation effect was measured through Trypan blue stain method in which cells surviving after treatment with the extract were stained by Trypan blue and counted. FIG. 7 shows the results of the experiment in which MCF-7 cells were treated with the 1 mg/ml concentration of the Chinese licorice root extracts using hexane, EtOAc, CHCI₃ and 70% MeOH as an extraction solvent for 72 hours and the number of the cells were counted. In all the extracts, apoptotic effect of cancer cells was observed. As shown in FIG. 7, the numbers of the cells were significantly reduced and thus, it was confirmed that the cells were killed.

### Experimental Example 3

### Immature Uterotrophic Assay

The 80% EtOH extract of Chinese licorice root which showed inhibitory effect in the cell proliferation assay using MCF-7 cell line was used to perform the immature uterotrophic assay (Kang, K. S. et al., Toxicology Letters, 118:109, 2000).

40 immature Sprague-Dawley rats, aged 18 days, were kept in a breeding environment of a temperature of 22±3° C., a relative humidity of 50±10%, a ventilation frequency of 10 to 12 times/hr, light time of 12 hours, an illumination intensity of 150 to 200 lux while being supplied with AIN-76A diet (per 1 kg, thiamine HCI 0.6 g, riboflavin 0.6 g, pyridoxine HCI 0.7 g, niacine 3.0 g, calcium pantothenate 1.6 g, folic acid 0.2 g, biotin 0.02 g, Vitamin B12 (0.1%) 1.0 g, Vitamin A palmitate (500,000 IU/g) 0.8 g, Vitamin D3 (400,000 IU/g) 0.25 g, Vitamin E acetate (500 IU/g) 10.0 g, menadion sodium bisulfite 0.08 g, fine powdery sucrose 981.15 g). Each rat was subcutaneously administered once daily with bisphenol A (40 mg/ml/100 g body weight) and the Chinese licorice root EtOH extract (10 mg or 50 mg/ml/100 g body weight) suspended in corn oil for 3 days. 24 hours after the last administration day, the animals were killed by cervical dislocation.

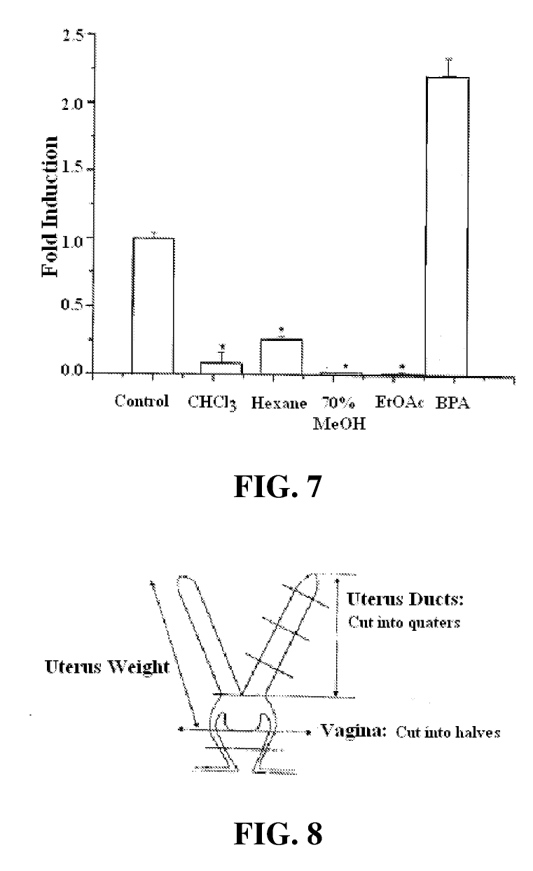

Upon autopsy, any liquid or fat in the uterus was entirely removed and the uterus was weighed and fixed with 10% formalin. By a conventional method, a tissue specimen was made and subjected to a histopathological test. The incision sites in the uterus and the vagina for the histopahological test are shown in FIG. 8. In particular, the height of the luminal epithelium of the uterus lumen was measured and the luminal epithelium of the vagina was carefully observed.

For statistical analysis of data with respect to body weight of test animals and the like which had been measured in the experiment, one-way ANOVA was performed to inspect significance between groups at p=0.05 level. When significance was approved, Dunnett's t-test was performed to inspect statistical significance between the control group and experimental group (p<0.05).

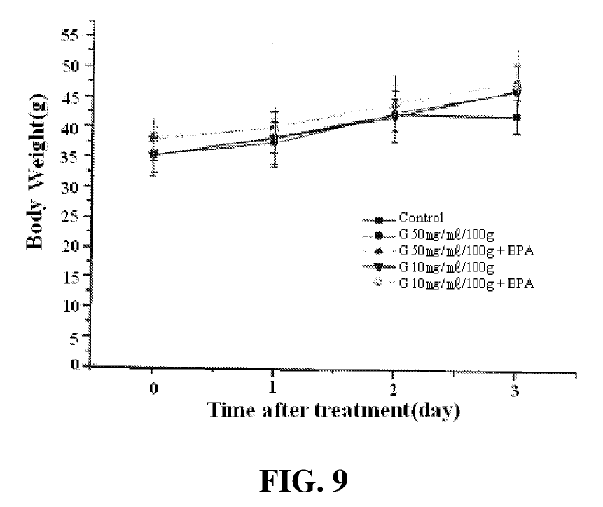

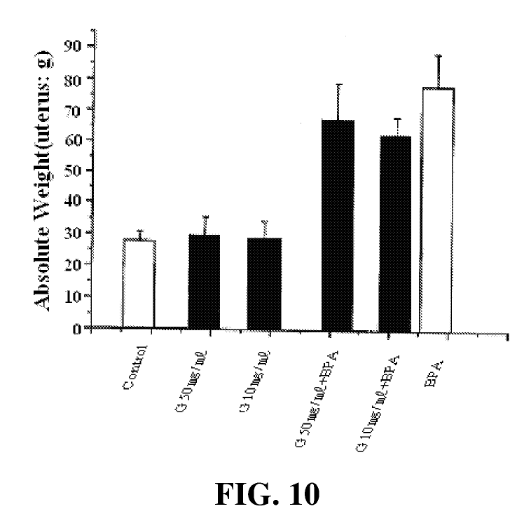

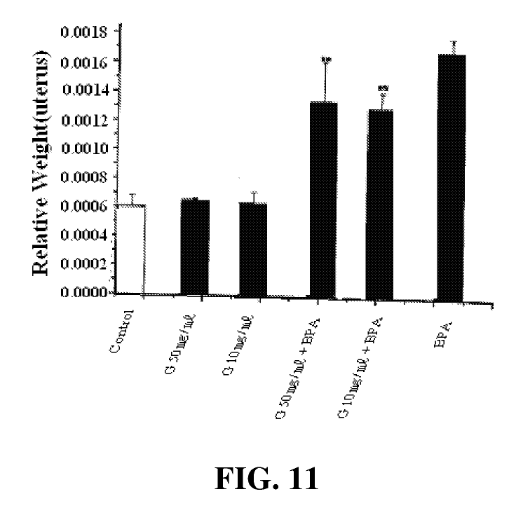

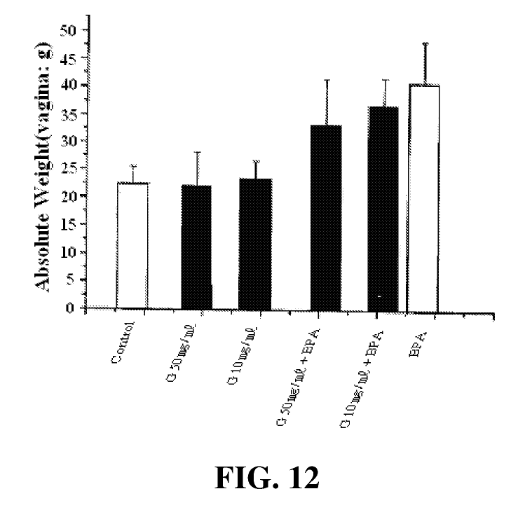

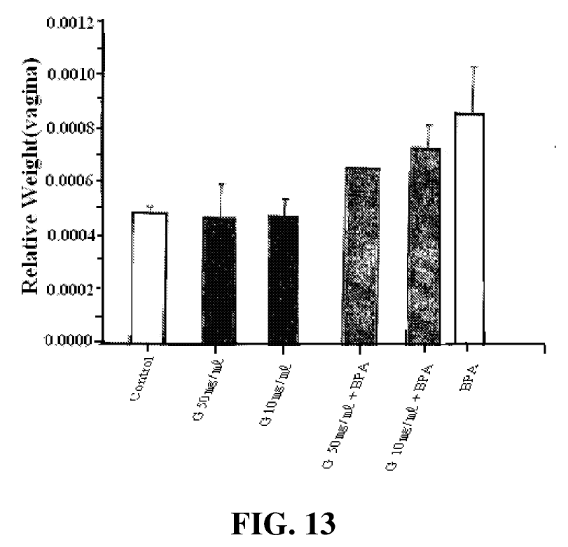

From the initial injection to the killing point, the animals were weighed every day. It was found that the body weight increase during the entire period of the experiment was normal as shown in FIG. 9. Also, the absolute weights of the removed uterus and the vagina did not show inhibitory effect as compared to the BPA treatment group, as shown in FIG. 10 and 12. However, in view of the relative weights determined by dividing the absolute weights of the removed uterus and vagina by the weight just before the autopsy, it was shown that the Chinese licorice root extracts had inhibitory effect on BPA in a concentration dependent manner, as illustrated in FIGS. 11 and 13, showing statistically significant change. While the absolute weight is merely data obtained by measuring an organ of an individual, the relative weight is a value obtained by calculating the weight of the organ of the individual, considering the body weight of the individual and thus, has significant meaning.

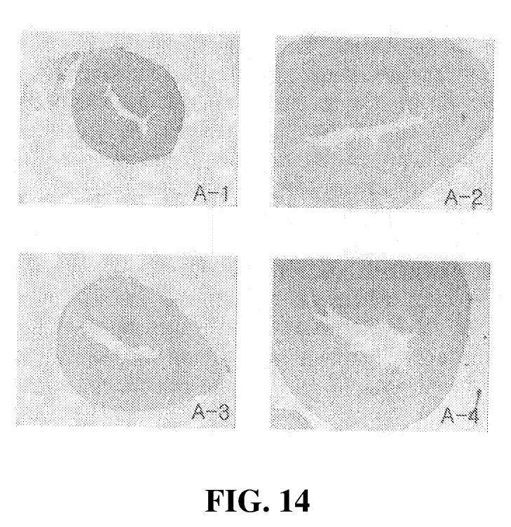

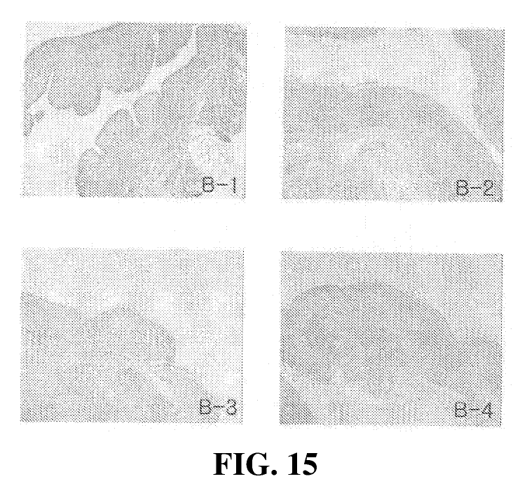

As a result of the histopathological test of the uterine epithelial cells and the vagina epithelial cells, it was shown that the thickness of the cells increased in the BPA treatment group, as compared to the non-treated control group. Also, when treated with the Chinese licorice root extract, the proliferation of the epithelial cells by BPA was inhibited in a concentration dependent manner at concentrations of 10 mg/ml and 50 mg/ml (FIG. 14 and FIG. 15). FIG. 14 shows tissue photographs of the uterine epithelial cells, in which A-1 is the non-treated control group, A-2 is the BPA treatment group, A-3 is the Chinese licorice root 50 mg/ml/100 g+BPA treatment group and A-4 is the Chinese licorice root 10 mg/ml/100 g+BPA treatment group. FIG. 15 shows tissue photographs of the vaginal epithelial cells, in which B-1 is the non-treated control group, B-2 is the BPA treatment group, B-3 is the Chinese licorice root 50 mg/ml/100 g+BPA treatment group and B-4 is the Chinese licorice root 10 mg/ml/100 g+BPA treatment group.

Formulation 1: Preparation of Pharmaceutical Formulation

Capsules were prepared by mixing the following ingredient and charging hard gelatin capsules with the mixture: Active ingredient (Chinese licorice root extract or isoliquiritin) 20 mg/capsule; dry starch 160 mg/capsule; and magnesium stearate 20 mg/capsule.

Formulation 2: Food Comprising the Chinese Licorice Root Extract or Isoliquiritin

Foods comprising the Chinese licorice root extract, liquiritin or isoliquiritin prepared in Example 1 and Example 2 according to the present invention were prepared as follows.

(1) Preparation of Wheat Flour Food

Wheat flour was mixed with 0.5% by weight of the Chinese licorice root extract or 0.05% by weight of liquiritin, and bread, noodles and the like were prepared using the resulting mixture to obtain foods for health promotion.

(2) Preparation of Dairy Products

Milk was mixed with 1% by weight of the Chinese licorice root extract or 0.1% by weight of isoliquiritin, and various dairy products such as butter and ice-cream were prepared using the milk mixture. In the case of preparation of cheese, the Chinese licorice root extract was added to curdled milk protein and in the case of preparation of yogurt, the extract was added to curdled milk protein obtained after fermentation.

(3) Preparation of Juice

1 to 5 g of the Chinese licorice root extract or 0.1 to 0.5 g of liquiritin was added to 1,000 Ml of tomato or apple juice to provide a juice for health promotion.

As described above, the composition comprising the Chinese licorice root extract and liquiritin or isoliquiritin isolated therefrom according to the present invention shows phytoestrogen activity and anti-tumor activity and thus can be usefully used in hormone replacement therapy and for prevention and treatment of cancers.

While the invention has been described with respect to particular features, aspects and embodiments, it will be recognized that the invention is not thus limited, but rather extends to and encompasses numerous variations, modifications and alternative embodiments, such as will suggest themselves to those of ordinary skill in the art, based on the disclosure herein.

Accordingly, the invention is intended to be broadly construed and interpreted, as encompassing all such variations, modifications and alternative embodiments, within the spirit and scope of the ensuing claims.

What is claimed is:

1. A phytoestrogen composition comprising at least one of liquiritin and isoliquiritin.

2. The phytoestrogen composition according to claim 1, wherein liquiritin and/or isoliquiritin is prepared by the steps of:
(a) adding an organic solvent to Chinese licorice root or a dehydrated product thereof, and extracting at temperature in a range of from 60° C. to 90° C., to yield an extract; and
(b) subjecting the extract to chromatographic separation including silica gel column chromatography and reverse phase column chromatography.

3. The composition according to claim 2, wherein the organic solvent comprises ethanol.

4. A method for prevention or treatment of a physiological condition comprises a disease state or condition selected from the group consisting of osteoporosis, cardiovascular diseases, menopausal symptoms and cancers, comprising administering to a subject in need thereof, an effective amount of a phytoestrogen composition comprising at least one of liquiritin and isoliquiritin.

5. The method according to claim 4, wherein the physiological condition comprises cancer.

6. A health-promoting food for prevention of menopausal symptoms and cancers, comprising at least one of liquiritin and isoliquiritin.

7. The health-promoting food according to claim 6, wherein liquiritin and/or isoliquiritin is present in an amount in a range of from 0.01 to 5 wt %, based on total weight of the food.

8. The method according to claim 5, wherein said cancer comprises breast cancer.

9. The method according to claim 5, wherein the physiological condition comprises menopausal symptoms.

10. The method according to claim 5, wherein the physiological condition comprises osteoporosis.

11. The method according to claim 5, wherein the physiological condition comprises cardiovascular disease.

12. The phytoestrogen composition according to claim 1, comprising liquiritin.

13. The phytoestrogen composition according to claim 1, comprising isoliquiritin.

14. The phytoestrogen composition according to claim 1, comprising liquiritin and isoliquiritin

15. The method according to claim 4, wherein said phytoestrogen composition comprises an effective amount of liquiritin.

16. The method according to claim 4, wherein said phytoestrogen composition comprises an effective amount of isoliquiritin.

17. The method according to claim 4, wherein said phytoestrogen composition comprises an effective amount of liquiritin and isoliquiritin.

18. The health-promoting food according to claim 6, comprising liquiritin.

19. The health-promoting food according to claim 6, comprising isoliquiritin.

20. The health-promoting food according to claim 6, comprising liquiritin and isoliquiritin.
